(19) United States
(12) Patent Application Publication
(10) Pub. No.: US 2023/0307093 A1
(43) Pub. Date: Sep. 28, 2023

(54) METHOD FOR PREDICTING DNA RECOMBINATION SITES BASED ON XGBOOST

(71) Applicant: Shanghai Institute of Technology, Shanghai (CN)

(72) Inventors: Zhendong Liu, Shanghai (CN); Yunxiang Liu, Shanghai (CN); Xi Chen, Shandong (CN); Ying Chen, Shanghai (CN)

(73) Assignee: Shanghai Institute of Technology, Shanghai (CN)

(21) Appl. No.: 18/151,485

(22) Filed: Jan. 9, 2023

(30) Foreign Application Priority Data
Jan. 11, 2022 (CN) 202210024162.3

(51) Int. Cl. G16B 20/30; G06F 17/11; G06F 18/214

(52) U.S. Cl. CPC G16B 20/30; G06F 17/11; G06F 18/214

(57) ABSTRACT

The present invention provides a method for preparing a transparent free-standing titanium dioxide nanotube array film. In the method, with the titanium foil as a substrate, the titanium dioxide nanotube array film is obtained by anode oxidation on the surface of the titanium foil. Upon high temperature annealing, the titanium dioxide nanotube array film naturally falls off to obtain the transparent free-standing titanium dioxide nanotube array film. The method according to the present invention features simple operations, saves time and cost. With the method, a completely strippable titanium dioxide nanotube array film may be prepared, and in addition, morphology of the titanium dioxide nanotube is not damaged. The free-standing and complete titanium dioxide nanotube array film facilitates transfer and post-treatment, has the feature of transparency and may be in favor of the applications to the studies such as photocatalysis and the like.

### CROSS-REFERENCE TO RELATED APPLICATION

This application claims the priority benefit of China Application Serial No. 202210024162.3, filed on Jan. 11, 2022. The entirety of the above-mentioned patent application is hereby incorporated by reference herein and made a part of this specification.

### BACKGROUND

### Technical Field

The present disclosure relates to the field of computational biology, mainly about a method for predicting DNA recombination sites, in particular to a method for predicting DNA recombination sites based on XGBoost.

### Description of Related Art

DNA recombination refers to the process that different DNA molecules are broken and connected to produce the exchange of DNA fragments and recombine to form new DNA molecules, which is one of the basic tools used in genetic engineering. The development of DNA recombination technology has greatly promoted the rapid development of molecular biology. Site-specific recombination is a kind of DNA recombination, which refers to the rearrangement of DNA sequences in the relative positions of DNA fragments, and does not depend on the homology of DNA sequences, but depends on the existence of DNA sequences that can be combined with certain enzymes. Studying the specific recombination sites of a bacterial integration subsystem can provide a new idea for the development of a recombination system.

attC is the main site for site-specific recombination in the integration subsystem. Previous studies have shown that tyrosine recombinase has high sequence homology requirements for the recombined attI sites, but the recombinase can effectively recombine the attC sites with highly variable sequences and structures. At the same time, the binding and recombination of integrase depends on three unpaired structural features of the attC sites: external helix bases (EHBs), an unpaired central spacer (UCS) and a variable terminal structure (VTS). Therefore, studying the correlation between the structure and function of the attC sites is helpful to solve the problem of restriction of recombination site sequences and develop a structure-specific DNA recombination system that does not depend on a consensus sequence or a similar sequence.

### SUMMARY

Aiming at the restriction problem of the site sequence level, the present disclosure provides a method for predicting DNA recombination sites based on XGBoost by XGBattCPred. XGBattCPred uses a data-driven method, focusing on attC sites of a bacterial integration subsystem, analyzing and quantifying the structural features of attC sites, constructing a regression prediction model by combining the structural data of sites with the XGBoost regression algorithm, constructing a high-precision prediction model according to the parameter optimization strategy, and using the feature importance measure to screen features to improve the design method of synthesizing sites. The object is to solve the problem that the current recombination site prediction experiment is time-consuming and low in efficiency and the problem of the sequence restriction in the site recombination process.

In order to achieve the above object, the present disclosure provides the following technical scheme: a method for predicting DNA recombination sites based on XGBoost, comprising the following steps:
(1) preprocessing an initial structural data set D= {D₁, D₂, ..., Dₙ} of attC sites, and performing screening, deletion and normalization on each feature Dᵢ (1≤i≤n) in the data set D, and obtaining the data set D′ through the above data preprocessing;
(2) for the data set D′ preprocessed in step (1), defining the threshold value of the attC site recombination rate as a, classifying the sites in the data set into positive sites (recombination rate ≥a) and negative sites (recombination rate < a), and adding a class column to the data set D′ to mark the samples, in which the positive sites are marked as 1 (class=1), and the negative sites are marked as 0 (class=0); screening positive and negative samples, and under-sampling the data set D′ to construct a balanced data set to obtain the data set D″; wherein the value range of a is [0.4-1];
(3) dividing the data set D″ obtained in step (2) according to the ratio M:N of the number of training sets to the number of verification sets, where M is the number of training sets in the data set D″ and N is the number of verification sets in the data set D″, so as to construct an initial XGBoost regression prediction model; wherein the value range of M:N is 1-6:1;
(4) optimizing parameters of the initial model obtained in step (3), wherein an Optuna framework is an efficient hyperparameter optimization framework; using the Optuna framework to perform iterative optimization training on the hyperparameters of the XGBoost regression model for b times and c rounds continuously; using k-fold cross-validation to select b groups of optimal hyperparameter combinations T={T₁, T₂, ..., Tₙ} (1≤n≤b), wherein the cross-validation score of each group of hyperparameters is calculated by the formula
$${\text{CV}}_{( \text{k)}} = {\text{Σ}}_{\text{i=1}}^{\text{k}} {\text{MSE}}_{1} ,$$
in which
$$\text{MSE =} \frac{1}{\text{m}} {\text{Σ}}_{\text{i=1}}^{\text{m}} {( {\text{y}}_{1} {\text{= y}}_{2}^{\hat{}} )}^{2}$$
is the mean square error, k means that the data set D″ is divided into k parts on average; the value range of b is [1-10], the value range of c is [50-200], and the value range of k is [5-10];
(5) using b groups of optimal hyperparameter combinations T obtained in step (4) to reconstruct the XGBoost regression prediction model W={W₁, W₂, ..., Wₙ} (1≤n≤b), respectively, dividing the data set D″ into a training set and a verification set at the ratio of M:N, inputting the training set into the optimized XGBoost regression model to train the model, and inspecting the performance of the model through the verification set;
(6) constructing an evaluation mechanism through the models obtained in step (4) and step (5), evaluating the performance of the model, and evaluating and predicting the performance of b regression models by the formula
$$\text{PCC} \text{=} \frac{{\sum }_{\text{i} = 1}^{\text{m}} \left({\text{y}}_{\text{i}} - \overline{\text{y}}\right) \left({\text{z}}_{\text{i}} - {\overline{\text{z}}}_{\text{i}}\right)}{\sqrt{\left[{\sum }_{\text{i} = 1}^{\text{m}} {\left({\text{y}}_{\text{i}} - {\overline{\text{y}}}_{\text{i}}\right)}^{2}\right] \left[{\sum }_{\text{i} = 1}^{\text{m}} {\left({\text{z}}_{\text{i}} - {\overline{\text{z}}}_{\text{i}}\right)}^{2}\right]}} ,$$
the formula
$$\text{MAE} \text{=} \frac{1}{\text{m}} {\sum }_{\text{i} = 1}^{\text{m}} \left(\left|{\text{y}}_{\text{i}} - {\text{z}}_{\text{i}}\right|\right) ,$$
the formula
$$\text{RMSE} \text{=} \frac{1}{m} {\sum }_{i = 1}^{m} \sqrt{{\left({y}_{i} - {z}_{i}\right)}^{2}}$$
and the formula
$$\text{varScore =} \frac{1}{\text{m}} {\text{Σ}}_{l = 1}^{m} [ 1 - \frac{{\text{Var(y}}_{\text{i}} - {\text{z}}_{\text{i}} )}{{\text{Var(y}}_{\text{i}} )} ] ,$$
where yᵢ and zᵢ represent the actual recombination rate and the predicted recombination rate, respectively, y̅ and z̅ are their average values, m is the total number of data points, and Var is the variance of each distribution;
(7) evaluating the evaluation index scores of the b regression models obtained in step (6) reasonably, and according to the standard:
$$\left\{{}_{i f n o t m e e t i n g t h e r e q u i r e m e n t s , r e - m o d e l i n g , o t h e r s}^{i f m e e t i n g r e q u i r e m e n t s , \text{PCC>0} \text{.81, MAE<0} \text{.093, RMSE<0} \text{.015, VarScore>0} \text{.65}}\right) ,$$
selecting the XGBoost regression prediction model Wᵢ with the highest precision as the final prediction model; inputting the data set D″ obtained in step (2) into the Wᵢ model meeting the requirements for training the model, and inputting the prediction set into the trained Wᵢ regression model to obtain the recombination rate of each point in the prediction set; (8) measuring the importance of the features according to the training prediction result output in step (7), scoring each feature in the recombination site feature sequence according to the importance acting on the prediction model as Rᵢ (1 i≤ in which
$${\sum }_{\text{i} = 1}^{\text{m}} {\text{R}}_{\text{i}} = 1 ,$$
q is the number of features in the data set D″ (1 ≤ q < n), and screening out the important features in the feature sequence according to the judgment:
$$\left\{\begin{matrix}i m p o r t a n t f e a t u r e s , {R}_{i} \geq 0.01 \\ b a s i c f e a t u r e s , {R}_{i} < 0.01\end{matrix} ;\right)$$
according to the score data of the output feature sequence, obtaining the important features that play a positive role in recombination, and obtaining the prediction model of improved recombination sites for improving the design of synthesizing the recombination sites.

Preprocessing the data set D in step (1) comprises the following steps:
(1-1) if for each Dᵢ (1≤i≤n), Dᵢⱼ (1≤j≤m) is all zeros, removing the feature Dᵢ;
(1-2) judging the variance of Dᵢ by the formula
$${\text{S}}^{2} = \frac{\left(\text{μ} - {\text{x}}_{1}\right) {}^{2} + \left(\text{μ} - {\text{x}}_{2}\right) {}^{2} + \left(\text{μ} - {\text{x}}_{3}\right) {}^{2} + … + \left(\text{μ} - {\text{x}}_{\text{m}}\right) {}^{2}}{\text{m}} ,$$
and removing the feature Dᵢ if S²_{Di}=0, where µ is the average of m values of the feature Dᵢ; the value range of m is [0-12,879];
(1-3) standardizing Dᵢ by the formula
$$\text{z =} \frac{\text{x-μ}}{\text{σ}} ,$$
where µ is the average of m values of Dᵢ, and σ is the standard deviation of m values of Dᵢ;
(1-4) normalizing Dᵢ linearly by the formula
$${\text{x}}_{\text{norm}} \text{=} \frac{{\text{x - x}}_{\text{min}}}{{\text{x}}_{\text{max}} {\text{- x}}_{\text{min}}} ,$$
and scaling the value of Dᵢ to [0,1], where Xₘᵢₙ is the minimum of m values of Dᵢ, and Xₘₐₓ is the maximum of m values of Dᵢ.

Preferably, in step (2), the value of a is 0.46, the positive site is marked as 1, and the negative site is marked as 0.

Preferably, in step (3), the value of M is 2, and the value of N is 1.

Preferably, in step (4), the value of b is 4, the value of c is 100, and the value of k is 5.

Compared with the prior art, the present disclosure has the following beneficial effects.

This algorithm constructs a high-precision prediction model for recombination sites. The important feature pairs screened according to the modeling results are effective supplements to the existing results, which can help improve the design method of recombination sites and realize more efficient recombination. The method for improving the design of synthesizing recombination sites is very effective, and the recombination rate between sites can be improved. Based on the idea of machine learning, the algorithm fully understands the correlation between the structure and function of recombination sites, and achieves a significant improvement in prediction efficiency. At the same time, aiming at the problem of sequence restriction, the important features are selected by screening the features of recombination sites, which can effectively improve the design method of recombination sites. Compared with the traditional random forest prediction algorithm, the present disclosure has higher efficiency, flexibility and visualization.

### BRIEF DESCRIPTION OF THE DRAWINGS

FIG. 1 is a flow chart of a method for predicting DNA recombination sites based on XGBoost.

FIG. 2 is a schematic structural diagram of attC recombination sites.

FIG. 3 is a schematic diagram of attCᵣ₀ folding structure used to construct a mutant library.

FIG. 4 is a score diagram of all features in a feature sequence.

### DESCRIPTION OF THE EMBODIMENTS

In order to clearly illustrate the technical scheme of the present disclosure, the present disclosure will be described below with reference to FIS. 1-4 through specific embodiments. The embodiments here are only used to explain the present disclosure, rather than limit the present disclosure.

It should be pointed out that the following detailed description is exemplary and is intended to provide further explanation of the present disclosure. Unless otherwise indicated, all technical and scientific terms used herein have the same meanings as commonly understood by those skilled in the art to which the present disclosure belongs.

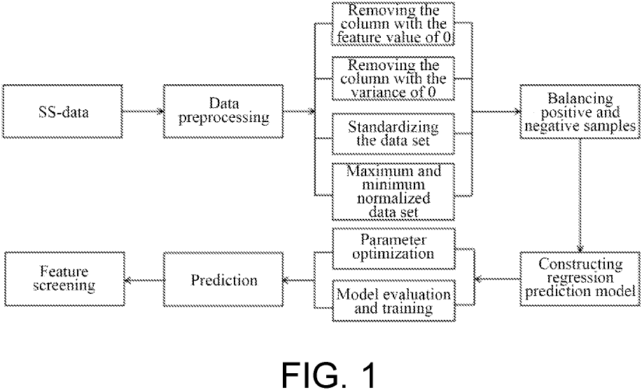

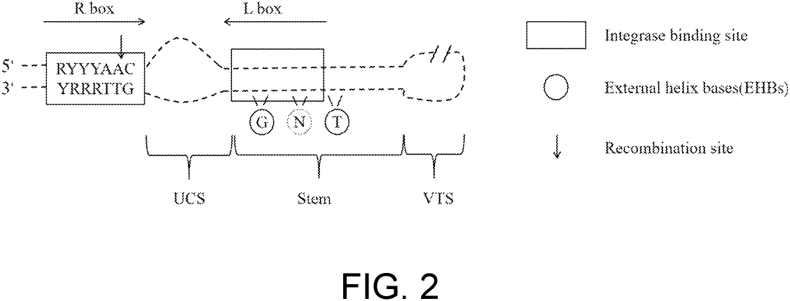

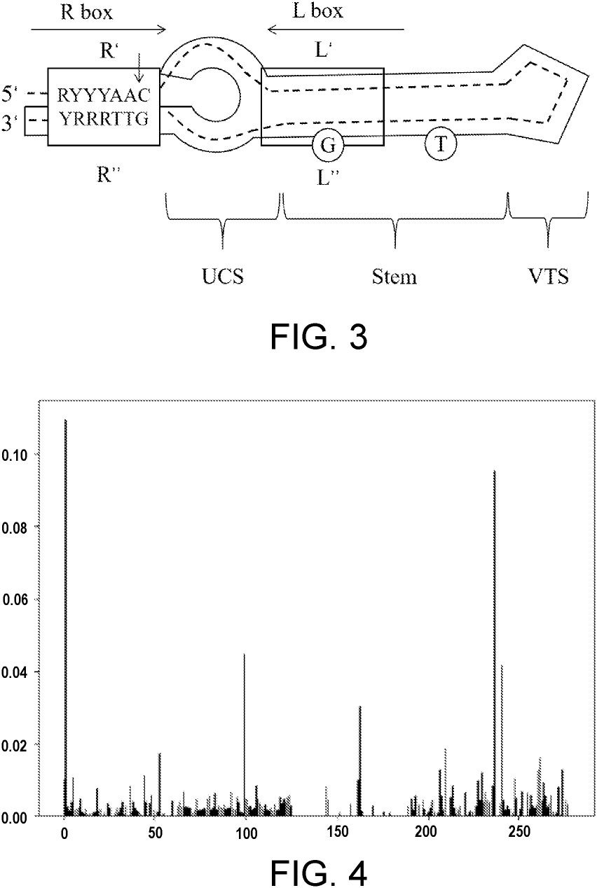

FIG. 1 shows the flow steps of the method for predicting DNA recombination sites by XGBattCPred. The DNA recombination site selected in this embodiment is the attC site of the bacterial integration subsystem. The structure diagram of the attC site is shown in FIG. 2. As the structure of this site is highly dependent on its function, a prediction model is established for the structural features of the site. It can be explained that the method is also applicable to other DNA recombination sites and genetic elements based on sequence features. In this embodiment, the database selects to access the attCᵣ₀ mutant library for analysis. The library comprises all the sequences of single mutation in the constant region of attCᵣ₀ site (as shown in FIG. 3) and the sequence containing all the possible combinations of two mutations.

XGBattCPred input file contains a txt-type file and an input-type file. The L1_listABCD_input_file.txt file is the structural feature data set D of 12,879 attCᵣ₀ mutants (including 9 global features and 283 basic features, and some data of the database are shown in Table 1). On the basis of this data set, the initial data is preprocessed. The attCFeatures.input file is a data set Z containing the structural data of 13 attC sites, and the final prediction model is used to output the recombination rate of the above sites.

TABLE 1

| attC sites | Features | | | | | | |
|---|---|---|---|---|---|---|---|
| | MFE_dG_u | MFE_freq_u | Hbond_n_u | base_6 | pos_entr_16_u | bp_proba_2_62_u | Output |
| 1 | 0.4674 | 0.1193 | 0.6667 | 0.5 | 0.0268 | 0.931 | 0.3474 |
| 16 | 0.5819 | 0.081 | 0.625 | 0.5 | 0.1258 | 0.8865 | 0.2606 |
| 26 | 0.7079 | 0.0814 | 0.5 | 0.5 | 0.2958 | 0.9046 | 0.1876 |
| 66 | 0.5189 | 0.2245 | 0.6389 | 0.5 | 0.0426 | 0.9947 | 0.1877 |
| 211 | 0.4044 | 0.0672 | 0.7222 | 0.5 | 0.2648 | 0.997 | 0.6342 |
| 552 | 0.4444 | 0.0592 | 0.7083 | 0.5 | 0.2719 | 0.969 | 0.2964 |

XGBattCPred output file contains an under-sampling-type file, a reg-type file and an output-type file. L1_listABCD_input_file.undersampling file is the data set D″ obtained by under-sampling the data set D′ and balancing the positive and negative samples, and the model is constructed on this basis; L1_listABCD_output_file.reg file is the score result of the model on each evaluation index, which is used to evaluate the performance of the model; attCFrequencies.output file is the recombination rate of each site in the output data set Z. The output of the XGBattCPred method is the recombination rate of attC sites predicted by the method and its feature score. The following are the specific steps of predicting DNA recombination sites:

As shown in FIG. 1, the present disclosure can be divided into the following three modules.

### 1. Initial Data Set Preprocessing Module

First, the data of the initial structure database is preprocessed to remove outliers and features. Then, the threshold of recombination rate is set. The positive and negative samples are marked, and a label column is added as a standard data set. According to the number of positive samples (i.e., positive site samples), the standard data set is under-sampled to establish a balanced data set.

### 2. Model Constructing Module

First, the initial prediction model is constructed by dividing the balanced data set obtained by preprocessing, and then an Optuna framework is used to train the hyperparameters of the model. The cross-validation score is used for evaluation in the parameter optimization process. The machine learn model is reconstructed according to a group of hyperparameters with the highest score obtained by screening.

### 3. Model Evaluation and Prediction Module

The reconstructed prediction models are scored, and PCC, MAE, RMSE and VarScore scores of different models are acquired. The model with the best score of each index is screened out as the final prediction model. The balanced data set is divided into a training set and a verification set which are input into the model obtained by screening for training. Taking the structural feature data of the site to be predicted as input, the recombination rate of the site is predicted.

### 4. Feature Measurement and Analysis Module

Taking the balanced data set as input, according to the results of the training set and the verification set, the score of the attC site structure feature sequence is obtained. The top 20 features with the highest scores are analyzed, which can narrow the scope for finding other important features and provide information support for traditional biochemical experiments.

As shown in FIG. 1, the steps of each module of this embodiment are as follows.

### 1. Initial Data Set Preprocessing Module

In this embodiment, the initial structure data set D= {D₁, D₂, ..., Dₙ} of attCᵣ₀ mutant is preprocessed, where D contains 12,879 data points and 292 feature items (including 9 global features and 283 basic features), namely Dᵢ (1≤i≤292) and Dᵢⱼ (1≤j≤12,879). Preprocessing Dᵢ (1≤i≤292) in data set D comprises the following steps.

(1-1) if for each Dᵢ, Dᵢⱼ (1≤j≤12,879) is all zeros, the feature Dᵢ are removed. In this embodiment, there are no feature items with all zeros in the data set D, so that no features are removed. At this time, the data set D contains 12,879 data points and 292 feature items.

(1-2) the variance of Dᵢ is judged by the formula

$${S}^{2} = \frac{{\left(μ - {x}_{1}\right)}^{2} + {\left(μ - {x}_{2}\right)}^{2} + {\left(μ - {x}_{3}\right)}^{2} + … + {\left(μ - {x}_{m}\right)}^{2}}{m} ,$$

and the feature Dᵢ is removed if S²_{Di}=0, where µ is the average of 12,879 values of the feature Dᵢ. In this embodiment, there are 14 features with variance of 0 in the data set D, which are: base_1, base_2, base_3, base_4, base_5, base_6, base_7, base_8, base_9, bp_proba_29_32_u, bp_proba_30_33_u, bp_proba_30_32_u, bp_proba_30_31_u, and bp_proba_31_32_u. The above features in the data set D are deleted. At this time, the data set D contains 12,879 data points and 278 feature items.

(1-3) Dᵢ is standardized by the formula

$$\text{z =} \frac{\text{x - μ}}{\text{σ}} ,$$

where µ is the average of 12,879 values of Dᵢ, and σ is the standard deviation of 12,879 values of Dᵢ. In this embodiment, i=1 is taken as an example. The average value of the feature Dᵢ=MFE_dG_u is 0.470240, and the standard deviation of the feature Dᵢ=MFE_dG_u is 0.134266. At this time, the data set D contains 12,879 data points and 278 feature items.

(1-4) Dᵢ is normalized linearly by the formula

$${\text{x}}_{\text{norm}} \text{=} \frac{{\text{x - x}}_{\text{min}}}{{\text{x}}_{\text{max}} {\text{- x}}_{\text{min}}} ,$$

and the value of Dᵢ is scaled to [0,1], where Xₘᵢₙ is the minimum of 12,879 values of Dᵢ, and Xₘₐₓ is the maximum of 12,879 values of Dᵢ. In this embodiment, i=2 is taken as an example. The maximum value of feature Dᵢ=Boltz_dG_u is 0.8585, and the minimum value is 0.0229. The preprocessed standard data set D′ is obtained, where D′ contains 12,879 data points and 278 feature items.

For the standard data set D′, the threshold value of the attC site recombination rate is defined as a=0.46, and the sites in the data set are classified into positive sites (recombination rate ≥0.46) and negative sites (recombination rate < 0.46). A class column is added to the data set D′ to mark the samples. The classification information of all samples in the data set D′ is obtained, that is, the positive sites are marked as 1 (class =1), and the negative sites are marked as 0 (class = 0). The positive and negative samples are screened in the data set D′. The data set D′ is under-sampled to construct a balanced data set to obtain a balanced data set D″. In this embodiment, the standard data set D′ contains 1762 positive samples and 11117 negative samples. In the data set D′, 1762 negative samples are randomly selected and combined with the positive samples to form a balanced data set D″. D″ contains 3524 data points and 279 feature items (adding feature item class).

### 2. Model Constructing Module

The initial XGBoost regression prediction model is constructed from the balanced data set D″ according to the ratio of the training set : the verification set =2:1. In this embodiment, the number of samples in the training set and the verification set is 2349 and 1175, respectively.

The parameters of the obtained initial model are optimized. Optuna framework is an efficient hyperparameter optimization framework. In this embodiment, the Optuna framework is used to perform iterative optimization training on the hyperparameters of the XGBoost regression model for 4 times and 100 rounds continuously; 5-fold cross-validation is used to select the optimal four groups of hyperparameter combinations T={T₁, T₂, T₃, T₄}. During each training, the training set and the verification set are extracted from the balanced data set D″ according to the ratio of 4: 1. In the experiment, the number of samples in the training set and the verification set is 2819 and 705, respectively. The cross-validation score of each group of hyperparameters is calculated by the formula

$${\text{CV}}_{\text{(k)}} {\text{= Σ}}_{\text{i=1}}^{\text{k}} {\text{MSE}}_{\text{i}} ,$$

in which

$$\text{MSE =} \frac{1}{\text{m}} {\text{Σ}}_{\text{i=1}}^{\text{m}} {{\text{(y}}_{1} {\text{= y}}_{\text{i}}^{\hat{}} )}^{\text{2}}$$

is the mean square error, k means that the data set D″ is divided into k parts on average. In this embodiment, after four rounds of parameter optimization, four groups of optimal hyperparameter combinationsT={T₁, T₂, T₃, T₄} are obtained, respectively. The XGBoost regression prediction model W={W₁, W₂, W₃, W₄} is reconstructed by using these four groups of hyperparameter combinations. The data set D″ is divided into a training set and a verification set at a ratio of 2: 1. The number of samples in the training set and the verification set is 2349 and 1175, respectively. The training set is input into the optimized XGBoost regression model to train the model, and the performance of the model is inspected by the verification set.

### 3. Model Evaluation and Prediction Module

An evaluation mechanism is constructed to evaluate the model performance of the reconstructed prediction model. In this embodiment, the performance of four regression models is evaluated by the formula

$$\text{PCC =} \frac{{\text{Σ}}_{\text{i=1}}^{\text{m}} {\text{(y}}_{\text{i}} {\text{-} \overline{\text{y}}}_{\text{i}} ) ( {\text{z}}_{\text{i}} {\text{-} \overline{\text{z}}}_{\text{i}} )}{\sqrt{[ {\text{Σ}}_{\text{i=1}}^{\text{m}} {{\text{(y}}_{\text{i}} {\text{-} \overline{\text{y}}}_{\text{i}} )}^{2} ] [ {\text{Σ}}_{\text{i=1}}^{\text{m}} {( {\text{z}}_{\text{i}} {\text{-} \overline{\text{z}}}_{\text{i}} )}^{2} ]}} ,$$

the formula

$$\text{MAE =} \frac{1}{\text{m}} {\text{Σ}}_{\text{i=1}}^{\text{m}} ( {\text{\textbar y}}_{\text{i}} - {\text{z}}_{\text{i}} \text{\textbar} ) ,$$

the formula

$$\text{RMSE =} \frac{1}{m} {\text{Σ}}_{\text{i=1}}^{m} \sqrt{{( {y}_{i} - {z}_{i} )}^{2}}$$

and the formula

$$\text{varScore =} \frac{1}{\text{m}} {\text{Σ}}_{\text{i=1}}^{\text{m}} [ 1 - \frac{{\text{Var(y}}_{\text{i}} {\text{-z}}_{\text{i}} )}{{\text{Var(y}}_{\text{i}} \text{)}} ] ,$$

where yᵢ and zᵢ represent the actual recombination rate and the predicted recombination rate, respectively, y̅ and z̅ are their average values, m is the total number of data points, and Var is the variance of each distribution.

The score of the model evaluation index is the intuitive performance of evaluating the performance of the model. The evaluation index scores of the above four regression models are reasonably evaluated. The scores of each model in this embodiment are shown in Table 2. According to the standard:

$$\begin{matrix}i f m e e t i n g r e q u i r e m e n t s , \text{PCC>0} \text{.81,} \text{MAE<0} \text{.093,} \text{RMSE<0} \text{.015,} \text{VarScore>0} \text{.65} \\ i f n o t m e e t i n g t h e r e q u i r e m e n t s , \text{re-modeling,} \text{others} ,\end{matrix}$$

the W₂ model with the highest precision is selected as the final prediction model of this example, which is named as XGBattCPred. As shown in Table 3, XGBattCPred is compared with decision tree regression, ridge regression, support vector regression and random forest regression algorithms, the model used in this embodiment has achieved good scores in four evaluation dimensions, which indicates the powerful performance of XGBattCPred.

TABLE 2

| Model | Evaluation Index | | | |
|---|---|---|---|---|
| | PCC | MAE | RMSE | VarScore |
| W₁ | 0.83 | 0.088 | 0.014 | 0.68 |
| W₁ | 0.84 | 0.086 | 0.013 | 0.70 |
| W₁ | 0.83 | 0.089 | 0.015 | 0.69 |
| W₁ | 0.81 | 0.092 | 0.015 | 0.66 |

TABLE 3

| Regression Method | Evaluation Index | | | |
|---|---|---|---|---|
| | PCC | MAE | RMSE | VarScore |
| Dicision tree | 0.66 | 0.124 | 0.029 | 0.32 |
| Ridge | 0.80 | 0.097 | 0.016 | 0.64 |
| Support vector | 0.78 | 0.100 | 0.016 | 0.61 |
| Random forest | 0.81 | 0.093 | 0.015 | 0.65 |
| XGBattCPred | 0.84 | 0.086 | 0.013 | 0.70 |

The balanced data set D″ is divided and input into the XGBattCPred model for training the model; the prediction set Z is input into the trained XGBattCPred to achieve high-precision prediction of the recombination rate of each site in the prediction set. In this embodiment, taking the third attC site in Z as an example, the recombination rate of the site output by the XGBattCPred model is 0.32013062.

The recombination rates of all sites in the data set Z output by the XGBattCPred model are shown in Table 4.

TABLE 4

| Site sequence | recombination rate of the predicted site |
|---|---|
| Seq1 | 0.3194243 |
| Seq2 | 0.3262864 |
| Seq3 | 0.32013062 |
| Seq4 | 0.32717258 |
| Seq5 | 0.3286602 |
| Seq6 | 0.3301046 |
| Seq7 | 0.32717258 |
| Seq8 | 0.32966286 |
| Seq9 | 0.31319225 |
| Seq10 | 0.3218595 |
| Seq11 | 0.28384495 |
| Seq12 | 0.28698277 |
| Seq13 | 0.37401083 |

### 4. Feature Measurement and Analysis Module

According to the prediction result output by the training of the XGBattCPred model, the importance of features is measured. Each feature in the recombination site feature sequence is scored according to the importance acting on the prediction model as Rᵢ (1≤i≤q), in which

$${\text{Σ}}_{\text{i=1}}^{\text{m}} {\text{R}}_{\text{i}} = \text{1,}$$

q=278 is the number (1 ≤ q < n) of features in the data set D″. The score of each feature in the attC site structure feature sequence output in this embodiment is shown in FIG. 4. The top 20 important features with the highest scores are selected according to the judgment:

$$\left\{\begin{matrix}i m p o r t a n t f e a t u r e s , {R}_{i} \geq 0.01 \\ b a s i c f e a t u r e s , {R}_{i} < 0.01\end{matrix} ,\right)$$

which are Boltz_dG_u, MFE_freq_u, MFE_dG_u, pos_entr_38_u, pos_entr_46_u, bp_proba_14_49_u, bp_proba_16_49_u, pos_entr_18_u, pos_entr_37_u, pos_entr_39_u, base_54, pos_entr_14_u, bp_proba_24_37_u, pos_entr_17_u, pos_entr_44_u, pfold, Boltz_diversity_u, pos_entr_10_u, pos_entr_12_u and dG_ratio_BOT_TOP_u.

Feature screening is very effective in improving the design method of synthesizing recombination sites. In this embodiment, the scores of feature sequences indicate that the recombination of attC sites is the result of multiple features, and most features play a positive role in the recombination of attC sites. Therefore, characterizing the top 20 features with the highest scores in the feature sequence can not only focus on the important feature range and avoid wasting time by blindly conducting experiments, but also provide strong data support for the next biochemical experiment test by analyzing the specific reasons why this group of features have higher scores. Once considerable experimental results are obtained, the design method of synthesizing recombination sites will be effectively improved, and the recombination rate among sites will be increased.

In this example, three global features (Boltz_dG_u, MFE_freq_u, MFE_dG_u) obtain higher scores, followed by the probability and position entropy of base pairing. Analyzing the regions where these features are located and the states in which these features can play a positive role in the recombination rate can help improve the method of synthesizing recombination sites. To verify the reliability of the features proposed in this example, this example uses the obtained 20 features to construct the data set V={V₁, V₂, ..., Vₙ}(1≤n≤20), and uses the data set V to reconstruct the XGBoost regression prediction model. The scores of the model in four evaluation index dimensions are PCC=0.85, MAE=0.87, RMSE=0.013 and VarScore=0.71, which indicates that the 20 important features proposed in this example have high precision.

Finally, it should be explained that the above is only a preferred embodiment of the present disclosure, and it is not intended to limit the present disclosure. Although the present disclosure has been described in detail with reference to the aforementioned embodiments, it is still possible for those skilled in the art to modify the technical schemes described in the aforementioned embodiments or equivalently replace some of the technical features. Any modification, equivalent substitution, improvement, etc. made within the spirit and principle of the present disclosure shall be included in the scope of protection of the present disclosure.

What is claimed is:

1. A predicting method of DNA recombination sites based on XGBoost, comprising the following steps:
(1) preprocessing an initial structural data set D= {D₁, D₂, ..., Dₙ} of attC sites, and performing screening, deletion and normalization on each feature Dᵢ in the data set D, where 1≤i≤n, and obtaining a data set D′ through the above data preprocessing;
(2) for the data set D′ preprocessed in step (1), defining a threshold value of a attC site recombination rate as a, classifying the sites in the data set into positive sites with recombination rate ≥a and negative sites with recombination rate < a, and adding a class column to the data set D′ to mark samples, in which the positive sites are marked as 1, class=1, and the negative sites are marked as 0, class = 0; screening positive and negative samples, and under-sampling the data set D′ to construct a balanced data set to obtain a data set D″; wherein the value range of a is [0.4-1];
(3) dividing the data set D″ obtained in step (2) according to a ratio M:N of a number of training sets to a number of verification sets, where M is the number of training sets in the data set D″ and N is the number of verification sets in the data set D″, so as to construct an initial XGBoost regression prediction model; wherein the value range of M:N is 1-6:1;
(4) optimizing parameters of the initial model obtained in step (3), wherein an Optuna framework is an efficient hyperparameter optimization framework; using the Optuna framework to perform iterative optimization training on the hyperparameters of the XGBoost regression model for b times and c rounds continuously; using k-fold cross-validation to select b groups of optimal hyperparameter combinations T={T₁, T₂, ..., Tₙ}, where 1≤n≤b, wherein the cross-validation score of each group of hyperparameters is calculated by the formula
$${\text{CV}}_{\left(\text{k}\right)} = {\sum }_{\text{i=1}}^{\text{k}} \text{MSE,}$$
in which
$$\text{MSE=} \frac{\text{1}}{\text{m}} {{\sum }_{\text{i=1}}^{\text{m}} \left({\text{y}}_{\text{i}} {\text{=y}}_{\text{i}}^{∧}\right)}^{2}$$
is the mean square error, k means that the data set D″ is divided into k parts on average; the value range of b is [1-10], the value range of c is [50-200], and the value range of k is [5-10];
(5) using b groups of optimal hyperparameter combinations T obtained in step (4) to reconstruct the XGBoost regression prediction model W={W₁, W₂, ..., Wₙ}, respectively, where 1≤n≤b, dividing the data set D″ into a training set and a verification set at the ratio of M:N, inputting the training set into the optimized XGBoost regression model to train the model, and inspecting the performance of the model through the verification set;
(6) constructing an evaluation mechanism through the models obtained in step (4) and step (5), evaluating the performance of the model, and evaluating and predicting the performance of b regression models by the formula
$$\text{PCC=} \frac{{\sum }_{\text{i} = 1}^{\text{m}} \left({\text{y}}_{\text{i}} - {\overline{\text{y}}}_{\text{i}}\right) \left({\text{z}}_{\text{i}} - {\overline{\text{z}}}_{\text{i}}\right)}{\sqrt{\left[{\sum }_{\text{i} = 1}^{\text{m}} {\left({\text{y}}_{\text{i}} - {\overline{\text{y}}}_{\text{i}}\right)}^{2}\right] \left[{\sum }_{i = 1}^{m} {\left({\text{z}}_{1} - {\overline{\text{z}}}_{1}\right)}^{2}\right]}} ,$$
the formula
$$\text{MAE=} \frac{\text{1}}{\text{m}} {\sum }_{\text{i} = 1}^{\text{m}} \left(\left|{\text{y}}_{\text{i}} - {\text{z}}_{\text{i}}\right|\right) ,$$
the formula
$$\text{RMSE} \text{=} \frac{\text{1}}{\text{m}} {\sum }_{\text{i} = 1}^{\text{m}} \sqrt{{\left({\text{y}}_{\text{i}} - {\text{z}}_{\text{i}}\right)}^{2}}$$
and the formula
$$\text{varScore} \text{=} \frac{\text{1}}{\text{m}} {\sum }_{\text{i} = 1}^{\text{m}} \left[1 - \frac{\text{Var} \left({\text{y}}_{\text{i}} - {\text{z}}_{\text{i}}\right)}{\text{Var} \left({\text{y}}_{\text{i}}\right)}\right] ,$$
where y
ᵢ and zᵢ represent an actual recombination rate and a predicted recombination rate, respectively, y̅ᵢ and z̅ᵢ are their average values, m is a total number of data points, and Var is a variance of each distribution;
(7) evaluating the evaluation index scores of the b regression models obtained in step (6) reasonably, and according to the standard:
$$\left\{\begin{matrix}i f m e e t i n g \text{} r e q u i r e m e n t s , \text{PCC>0} \text{.81,} \text{MAE<0} \text{.093,RMSE<0} \text{.015,} \text{VarScore} > 0.65 \\ i f n o t m e e t i n g t h e r e q u i r e m e n t s , r e - m o d e l i n g , o t h e r s\end{matrix}\right) ,$$
selecting the XGBoost regression prediction model W
ᵢ with the highest precision as the final prediction model; inputting the data set D″ obtained in step (2) into the Wᵢ model meeting the requirements for training the model, and inputting the prediction set into the trained Wᵢ regression model to obtain the recombination rate of each point in the prediction set;
(8) measuring the importance of the features according to the training prediction result output in step (7), scoring each feature in the recombination site feature sequence according to the importance acting on the prediction model as Rᵢ, where 1≤i≤q, in which
$${\sum }_{\text{i} = 1}^{\text{n}} {R}_{i} = 1 ,$$
q is the number of features in the data set D″, where 1 ≤ q < n, and screening out the important features in the feature sequence according to the judgement:
$$\left\{\begin{matrix}i m p o r t a n t f e a t u r e s , {R}_{i} \geq 0.01 \\ b a s i c f e a t u r e s , {R}_{i} < 0.01\end{matrix} ;\right)$$
according to the score data of the output feature sequence, obtaining the important features that play a positive role in recombination, and obtaining the prediction model of improved recombination sites for improving the design of synthesizing the recombination sites.

2. The predicting method according to claim 1, wherein preprocessing the data set D in step (1) comprises the following steps:
(1-1) if for each Dᵢ, 1≤i≤n, Dᵢⱼ, 1≤j≤m, is all zeros, removing the feature Dᵢ;
(1-2) judging the variance of Dᵢ by the formula
$${\text{S}}^{2} = \frac{{\left(\text{μ−} {\text{x}}_{1}\right)}^{2} + {\left(\text{μ−} {\text{x}}_{2}\right)}^{2} + {\left(\text{μ−} {\text{x}}_{3}\right)}^{2} + … + {\left(\text{μ−} {\text{x}}_{\text{m}}\right)}^{2}}{\text{m}} ,$$
and removing the feature D
ᵢ if S²_{Di}=0, where µ is the average of m values of the feature Dᵢ; the value range of m is [0-12,879];
(1-3) standardizing Di by the formula
$$\text{Z} = \frac{\text{x} - \text{μ}}{σ} ,$$
where µ is the average of m values of D
ᵢ, and σ is the standard deviation of m values of Dᵢ;
(1-4) normalizing Dᵢ linearly by the formula
$${\text{X}}_{\text{norm}} = \frac{\text{X} - {\text{X}}_{\text{min}}}{{\text{X}}_{\text{max}} - {\text{X}}_{\text{min}}} ,$$
and scaling the value of D
ᵢ to [0,1], where Xₘᵢₙ is the minimum of m values of Dᵢ, and Xₘₐₓ is the maximum of m values of Dᵢ.

3. The predicting method according to claim 1, wherein in step (2), the value of a is 0.46, the positive site is marked as 1, and the negative site is marked as 0.

4. The predicting method according to claim 1, wherein in step (3), the value of M is 2, and the value of N is 1.

5. The predicting method according to claim 1, wherein in step (4), the value of b is 4, the value of c is 100, and the value of k is 5.

6. The predicting method according to claim 1, wherein in step (7), the number of decision trees of the XGBoost regression algorithm is 800, and the maximum depth of the trees is 4.
